SEQUENCE LISTING (1 sequence)

SEQ ID NO: 1 - DNA, 1429 bp, Artificial Sequence
  It is synthesized
cttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgactgcatgtatag

(19) United States
(12) Patent Application Publication
(10) Pub. No.: US 2023/0371525 A1
(43) Pub. Date: Nov. 23, 2023

(54) BACILLUS ALTITUDINIS AND APPLICATION OF ACTIVE SUBSTANCE COMPOUND SOLUTION AND MICROBIAL INOCULUM THEREOF IN CONTROL OF ROOT-KNOT NEMATODE DISEASE

(71) Applicant: JIANGSU ZIBAINONG ECOLOGICAL AGRICULTURE CO., LTD, Xuzhou (CN)

(72) Inventors: Bo ZHOU, Xuzhou (CN); Jianyu WANG, Xuzhou (CN); Peng ZHAO, Xuzhou (CN); Fengyuan YU, Xuzhou (CN); Hui WANG, Xuzhou (CN); Xingqiang GUO, Xuzhou (CN); Jinzhong FU, Xuzhou (CN)

(21) Appl. No.: 18/041,805

(22) PCT Filed: Aug. 18, 2020
(86) PCT No.: PCT/CN2020/109784

§ 371 Date: Feb. 16, 2023

(51) Int. Cl. A01N 63/22; C12N 1/20; C12P 7/54; C12P 7/16; A01P 5/00

(52) U.S. Cl. CPC A01N 63/22; C12N 1/205; C12P 7/54; C12P 7/16; A01P 5/00; C12R 2001/07

(57) ABSTRACT

Disclosed is Bacillus altitudinis and application of an active substance compound solution and a microbial inoculum thereof in control of root-knot nematode disease, where Bacillus altitudinis AMCC 101084 has been deposited at China General Microbiological Culture Collection Center (CGMCC, No. 3, Yard 1, Beichen West Road, Chaoyang District, Beijing, China) on Aug. 16, 2018 with deposit number CGMCC No. 16308. The microbial inoculum of the present invention has a significant control effect on root-knot nematodes.

### FIELD OF THE INVENTION

The present invention relates to Bacillus altitudinis and application of an active substance compound solution and a microbial inoculum thereof in control of root-knot nematode disease, belonging to the technical field of microbiology.

### BACKGROUND OF THE INVENTION

Root-knot nematode disease is a common soil-borne disease caused by root-knot nematodes, which has become one of the important factors restricting the development of agriculture in the world. According to statistics, the global annual loss due to root-knot nematode disease is up to 157 billion dollars. The main symptoms of root-knot nematode disease are the formation of nodules of different sizes in the roots of plants. The diseased plants show no obvious symptoms in the early stage, similar to the symptoms of insufficient water and fertilizer. Once found, it is the later stage of the disease, showing an explosive incidence, plant death, or no production.

The use of chemical agents to control root-knot nematodes dominates the current agricultural production. The chemical pesticides used to control root-knot nematodes are known as nematicides, which are divided into fumigants and non-fumigants. In the past decades, chemical nematicides have been welcomed by farmers with their economic and quick-acting characteristics. However, with the enhancement of people's awareness of environmental protection and the national emphasis on green agriculture and healthy agriculture, the use of chemical nematicides is gradually limited due to its high residue, high toxicity, high risk and other disadvantages, and the vast majority of widely used highly toxic nematicides such as fenamiphos, isosalfos-methyl, carbofuran, methomyl, methomyl, aldicarb, methyl bromide and so on have been banned.

With people's attention to root-knot nematode disease and deepening research, some chemical control methods have been explored, and biological control, as one of them, has become a hot research of root-knot nematode control. Biological control of root-knot nematodes refers to killing or inhibiting root-knot nematodes with their natural enemies or their metabolites to alleviate the occurrence of root-knot nematode disease. Biological control has been paid much attention to at the industrial level because of its environmental friendliness, obvious effect, no residue and safety to human and livestock. At present, more than 700 species of nematode-eating fungi have been discovered and reported. Bacteria such as Pseudomonas, Bacillus, Pasteurella and other genera have also been confirmed to have good nematicidal effect. New nematicidal compounds and genes have also been discovered and reported.

Although the research on biocontrol bacteria is more and more, but the proportion of registered and practical application in agricultural production is still very small, and the strains are relatively single. The important reason is that the understanding, research, development and utilization of nematode biocontrol bacteria is still in an initial stage, which fails to meet the practical requirements of biological control of nematode diseases. In the application of biocontrol bacteria, the research results only stay at an experimental level. In addition, the research and development of efficient fermentation process and dosage form with biocontrol bacteria as the main active ingredients are relatively lag, which results in a small number of microbial inoculum products of biocontrol bacteria for industrial production and large-scale application.

Although there are a few reports on the nematicidal activity of Bacillus altitudinis, the researches on Bacillus altitudinis are not deep enough with single fermentation and dosage form and low control efficiency, and there is no research on the nematicidal active substances of Bacillus altitudinis.

Chinese patent document CN108865946 A (application number: 201810799951.8) disclosed Bacillus altitudinis and application in control of root-knot nematodes of tomatoes. When the bacterial suspension of Bacillus altitudinis CGMCC No. 15213 acts on the second-stage juvenile of root-knot nematodes, the action time is long, being above 12 hours. The application experimental data of the strain shows that each tomato is irrigated with 250 mL liquid every time, totally irrigating the root twice, where the liquid is 10 times of liquid of the solid fermentation culture of Bacillus altitudinis CGMCC No. 15213, the viable count of the solid fermentation culture is 7 billion/g, and the solid fermentation culture of Bacillus altitudinis CGMCC No. 15213 applied per tomato is 50 g, namely, the viable count applied per tomato is 350 billion. The tomato variety used is “Xifen No. 3”, which is about 4500 plants per mu. Even when 2000 strains are cultivated per mu, the amount of the solid fermentation culture of Bacillus altitudinis CGMCC No. 15213 per mu is 100 kg according to the above experimental value, and the viable count reaches 7×10⁵ billion/mu. The use of Bacillus altitudinis CGMCC No. 15213 disclosed in the present invention for controlling nematodes is too large per mu to be popularized.

### SUMMARY OF THE INVENTION

In view of the deficiencies of the prior art, the present invention provides Bacillus altitudinis and application of an active substance compound solution and a microbial inoculum thereof, which can be used for biological control of root-knot nematode disease.

The technical solutions of the present invention are as follows:

Bacillus altitudinis AMCC 101084 has been deposited at China General Microbiological Culture Collection Center (CGMCC, No. 3, Yard 1, Beichen West Road, Chaoyang District, Beijing, China) on Aug. 16, 2018 with deposit number CGMCC No. 16308.

Bacillus altitudinis AMCC 101084, abbreviated as AMCC 101084 and numbered AMCC1040, is isolated from plant rhizosphere soil.

A culture method I of the AMCC 101084 comprises the following steps:
(1) activating and culturing the AMCC 101084 on a solid medium plate to prepare an activated strain;
(2) inoculating the activated strain prepared in step (1) into a seed liquid medium for cultivation to prepare a seed solution;
(3) inoculating the seed solution prepared in step (2) into a fermentation liquid medium for cultivation to prepare a primary fermentation broth; and
(4) inoculating the primary fermentation broth prepared in step (3) into a fermentation liquid medium for cultivation to prepare a fermentation broth.

According to the preferred of the present invention, in the step (1), the solid medium is a Luria-Bertani (LB) solid medium under an activation condition at 37° C. for 24 hours.

According to the preferred of the present invention, in the step (2), the seed liquid medium is an LB liquid medium under a culture condition of 37° C. and 180 rpm for 18 hours.

According to the preferred of the present invention, in the step (3), an inoculation amount is 5 to 10% by volume under a culture condition of 37° C. and 180 rpm for 10 hours.

According to the preferred of the present invention, in the step (4), an inoculation amount is 5 to 10% by volume under a culture condition of 37° C. and 180 rpm for 24 hours.

According to the preferred of the present invention, in the step (3) or (4), according to mass fraction, components of the fermentation liquid medium are as follows: 1.5% corn flour, 2.0% soybean meal, 2.1% bran, 0.03% KH₂PO₄, 0.01% K₂HPO₄, 0.01% CaCl₂), balance water, and pH 7.0 to 7.2.

According to the preferred of the present invention, in the step (4), a cell concentration of the prepared fermentation broth is (0.2 to 1.0)×10⁹ CFU/mL.

A culture method II of the AMCC 101084 comprises the following steps:
a. activating and culturing the AMCC 101084 on an LB solid medium plate to prepare an activated strain;
b. inoculating the activated strain prepared in step a into a Beef Prptone Yeast (BPY) liquid medium for cultivation to prepare a seed solution; and
c. inoculating the seed solution prepared in step b into a BPY liquid medium for cultivation to prepare a fermentation broth.

According to the preferred of the present invention, in the step a, an activation condition is at 37° C. for 12 hours.

According to the preferred of the present invention, in the step b, a culture condition is at 37° C. and 200 rpm for 12 hours.

According to the preferred of the present invention, in the step c, an inoculation amount is 1% and a culture condition is at 37° C. and 200 rpm for 48 hours.

According to the preferred of the present invention, in the step b or c, components per liter of the BPY liquid medium are as follows:

10.0 g peptone, 5.0 g yeast extract, 5.0 g beef extract, 10.0 g glucose, 5.0 g sodium chloride, balance water, and pH 7.0.

According to the preferred of the present invention, in the step c, a cell concentration of the prepared fermentation broth is (0.3 to 1.1)×108 CFU/mL.

The above-mentioned AMCC 101084 is used for controlling root-knot nematodes.

According to the preferred of the present invention, the above-mentioned AMCC 101084 is used for controlling root-knot nematodes of tomatoes and/or ginger.

The fermentation broth prepared by the above-mentioned culture method I or culture method II of AMCC 101084 is used for controlling root-knot nematodes.

According to the preferred of the present invention, the above-mentioned fermentation broth is used for controlling root-knot nematodes of tomatoes and/or ginger.

According to the preferred of the present invention, liquid of the above-mentioned fermentation broth without thallus is used for controlling root-knot nematodes.

According to the preferred of the present invention, the above-mentioned fermentation broth is applied at a dilution of 1000 times.

Further preferably, the above-mentioned fermentation broth is used for controlling root-knot nematodes of tomatoes and/or ginger in an amount of 20 L per mu.

The above-mentioned AMCC 101084 is used for producing one or more of 2,3-butanedione, acetic acid, acetoin, 3-methyl-1-butanol, 2-isopropoxy ethylamine, 2,3-butanediol, 3-methyl-butanoic acid, 2-methyl-butanoic acid, 2-ethyl hexanol, and n-octanoic acid.

A nematicidal active substance compound solution comprises one or more of 2,3-butanedione, acetic acid, acetoin, 3-methyl-1-butanol, 2-isopropoxy ethylamine, 2,3-butanediol, 3-methyl-butanoic acid, 2-methyl-butanoic acid, 2-ethyl hexanol, and n-octanoic acid.

According to the preferred of the present invention, the compound solution, in terms of mass fraction, comprises 0.1% to 0.5% 2,3-butanedione, 0.1% to 0.4% acetic acid, 0.5% to 1.0% acetoin, 0.1% to 0.5% 3-methyl-1-butanol, 0.3% to 1.0% 2-isopropoxy ethylamine, 0.1% to 0.5% 2,3-butanediol, 0.1% to 0.4% 3-methyl-butanoic acid, 0.1% to 0.3% 2-methyl-butanoic acid, 0.1% to 0.5% 2-ethyl hexanol, and 0.01% to 0.1% n-octanoic acid, with the balance being water.

Further preferably, the compound solution, in terms of mass fraction, comprises 0.3% 2,3-butanedione, 0.2% acetic acid, 0.7% acetoin, 0.3% 3-methyl-1-butanol, 0.8% 2-isopropoxy ethylamine, 0.4% 2,3-butanediol, 0.2% 3-methyl-butanoic acid, 0.2% 2-methyl-butanoic acid, 0.5% 2-ethyl hexanol, and 0.05% n-octanoic acid, with the balance being water.

### Advantageous Effects

1. Fermentation supernatant of AMCC 101084 involved in the present invention shows 100% lethality to second-stage juvenile of Meloidogyne incognita in a nematicidal experiment, and the action time is short, with only 1.5 hours required to achieve the 100% lethality.
2. After 3 days of treatment of nematode-infected soil with AMCC 101084 liquid microbial inoculum of the present invention, ginger is planted. In the later stage, the microbial inoculum is no longer applied, and the amount of fermentation broth is less. Compared with the blank control, the bark cracking area of ginger to which the microbial inoculum is applied is significantly reduced, where the root-knot nematode disease of ginger is commonly known as ginger bark cracking disease, effectively improving the product quality of ginger. The microbial inoculum of the present invention has a significant control effect on root-knot nematodes.
3. After 3 days of treatment of nematode-infected soil with active substance compound solution of the present invention, tomatoes are transplanted. Root-knots of the tomatoes decrease by 65.86% after planting for 45 days, and population density of nematodes decreases by 73.24%.

### BRIEF DESCRIPTION OF THE DRAWINGS

FIG. 1 is a phylogenetic tree of AMCC 101084;

FIG. 2 is a curve graph of root-knot nematode lethality corresponding to action time of supernatant of fermentation broth II in Embodiment 2;

FIG. 3 is total ion current of volatile substances of AMCC 101084;

FIG. 4 are observation photos showing inhibitory effects of a fermentation broth of AMCC 101084 on root-knot nematodes of ginger;

FIG. 5 is an observation photo showing ginger bark cracking in a blank control group at harvest time; and

FIG. 6 is an observation photo showing ginger bark cracking to which a microbial inoculum is applied at harvest time.

### DETAILED DESCRIPTION OF THE EMBODIMENTS

The technical solutions of the present invention will be further illustrated with reference to the following embodiments and drawings, but the scope of the present invention is not limited thereto.

The experimental methods in the following embodiments are conventional unless otherwise specified.

The test materials used in the following embodiments, unless otherwise specified, are purchased from conventional biochemical manufacturers.

For the quantitative experiments in the following embodiments, three replicates are set up and the results are averaged.

### Embodiment 1

Isolation and Identification of AMCC 101084

A nematicidal strain, named strain AMCC 101084 and numbered AMCC1040, was isolated from plant rhizosphere.

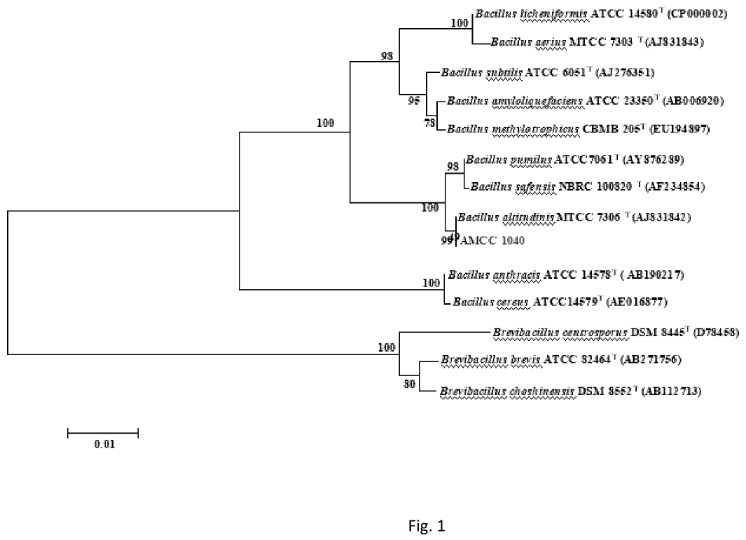

The 16S rDNA sequence of the strain AMCC 101084 was shown in SEQ ID No. 1, and its taxonomic status is in the same branch as Bacillus altitudinis (see FIG. 1). Physiological and biochemical experiments (see Table 1) showed that the strain had the same taxonomic status as Bacillus altitudinis.

The strain AMCC 101084 was identified as Bacillus altitudinis and has been deposited at China General Microbiological Culture Collection Center (CGMCC, No. 3, Yard 1, Beichen West Road, Chaoyang District, Beijing, China) on Aug. 16, 2018 with deposit number CGMCC No. 16308.

TABLE 1
Physiological and biochemical experiments

| Substance | Results | Substance | Results | Substance | Results |
|---|---|---|---|---|---|
| Dextrin | − | D-Sorbitol | − | pH 6 | ++ |
| D-Maltose | − | D-Arabitol | − | pH 5 | − |
| D-Trehalose | + | myo-Inositol | − | 1% NaCl | ++ |
| Gentiobiose | ++ | Glycerol | ++ | 4% NaCl | ++ |
| Sucrose | + | D-Glucose-6- | − | 8% NaCl | ++ |
| | | PO₄ | | | |
| D-Turanose | − | D-Fructose-6- | + | 1% | ++ |
| | | PO₄ | | Sodium | |
| | | | | Lactate | |
| Stachyose | − | D-Aspartic | − | Fusidic | − |
| | | Acid | | Acid | |
| D-Raffinose | + | D-Serine | − | D-Serine | − |
| α-D-Lactose | − | α-D-Glucose | ++ | Rifamycin SV | − |
| D-Melibiose | + | Glycyl-L-Prolin | − | Lincomycin | − |
| β-Methyl-D-Glucoside | ++ | L-Alanine | + | Guanidine HCl | − |
| D-Salicin | ++ | L-Arginine | + | Vancomycin | − |
| D-Fructose | ++ | D-Glucuronic | + | Nalidixic | − |
| | | Acid | | Acid | |
| D-Galactose | + | D-Cellobiose | + | Lithium | ++ |
| | | | | Chloride | |
| 3-Methyl Glucose | − | L-Lactic Acid | ++ | Potassium | ++ |
| | | | | Tellurite | |
| D-Fucose | − | Citric Acid | − | Aztreonam | ++ |
| L-Fucose | − | Stachyose | − | D-Malic | − |
| | | | | Acid | |
| L-Rhamnose | − | Acetic Acid | + | D-Mannitol | ++ |
| Inosine | − | Tween 40 | + | Niaproof | − |
| | | | | 4 | |
| N-Acetyl-D- | + | N-Acetyl-β- | − | N-Acetyl-D- | − |
| Glucosamine | | DMannosamine | | Galactosamine | |
| D-Mannose | + | Troleandomycin | − | Minocycline | − |
| L-Aspartic | ++ | L-Glutamic | ++ | L-Histidine | − |
| Acid | | Acid | | | |
| L-Pyroglutamic | + | L-Serine | + | Niaproof | − |
| Acid | | | | 4 | |
| Pectin | ++ | D-Gluconic | + | D-Glucuronic | − |
| | | Acid | | Acid | |
| Glucuronamide | + | Mucic Acid | − | Quinic | ++ |
| | | | | Acid | |
| D-Saccharic | − | Tetrazolium | + | Tetrazolium | + |
| Acid | | Violet | | Blue | |
| L-Malic | ++ | Bromo- | + | α-Hydroxy- | − |
| Acid | | Succinic Acid | | Butyric | |
| | | | | Acid | |
| Formic Acid | − | Sodium | ++ | Sodium | + |
| | | Butyrate | | Bromate | |

Note:
++ means positive, + means borderline, − means negative.

### Embodiment 2

Culture and In Vitro Nematicidal Experiment of Strain AMCC 101084

A culture method I of AMCC 101084 comprised the following steps.
(1) The strain was deposited on a slant, delineated in three zones on an LB solid medium plate and cultured at 37° C. for 24 hours to prepare an activated strain.
(2) The activated strain prepared in step (1) was inoculated into an LB liquid medium for cultivation at 37° C. and 180 rpm for 18 hours to prepare a seed solution.
(3) The seed solution prepared in step (2) was inoculated into a fermentation liquid medium with an inoculation amount of 5% by volume for cultivation under a culture condition of 37° C. and 180 rpm for 10 hours to prepare a primary fermentation broth.
(4) The primary fermentation broth prepared in step (3) was inoculated into a fermentation liquid medium with an inoculation amount of 7% by volume for cultivation under a culture condition of 37° C. and 180 rpm for 24 hours to prepare a fermentation broth I.

In the step (3) or (4), according to mass fraction, components of the fermentation liquid medium were as follows: 1.5% corn flour, 2.0% soybean meal, 2.1% bran, 0.03% KH₂PO₄, 0.01% K₂HPO₄, 0.01% CaCl₂), balance water, and pH 7.0 to 7.2.

The viable count of the fermentation broth I was (0.2 to 1.0)×10⁹ CFU/mL.

A culture method II of the AA/ICC 101084 comprised the following steps.
(1) The strain was deposited on a slant, delineated in three zones on an LB solid medium plate and cultured at 37° C. for 12 hours to prepare an activated strain.
(2) The activated strain prepared in step (1) was inoculated into a BPY liquid medium (50 mL/250 mL) at 37° C. and 200 rpm for 12 hours to prepare a seed solution.
(3) The seed solution was inoculated into a BPY medium (200 mL/500 mL) with an inoculation amount of 1% at 37° C. and 200 rpm for 48 hours to prepare a fermentation broth II.

The viable count of the fermentation broth II was (0.3 to 1.1)×108 CFU/mL.

Components per liter of the LB medium were as follows:
10.0 g peptone, 5.0 g yeast extract, 5.0 g sodium chloride, 20.0 g agar powder, balance water, and pH 7.0, sterilized at 121° C. for 20 minutes.

Components per liter of the BPY medium were as follows:
10.0 g peptone, 5.0 g yeast extract, 5.0 g beef extract, 10.0 g glucose, 5.0 g sodium chloride, balance water, and pH 7.0, sterilized at 121° C. for 20 minutes.

Preparation of supernatant of fermentation broth II: the fermentation broth was distributed into centrifuge tubes of 100 mL and placed into a high-speed refrigerated centrifuge at 12000 rpm, centrifuging at 4° C. for 10 minutes, sucking the supernatant, and storing in a refrigerator at 4° C. for later use.

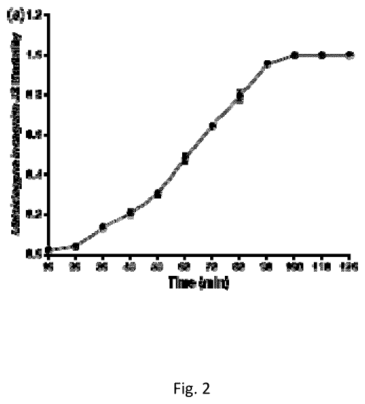

Activity assay: 100 μL of nematode suspension (about 200 nematodes), 100 μL of supernatant of fermentation broth II of to-be-tested bacteria and 300 μL of sterile water were added into a 24-well plate, gently blowing and mixing well with a pipettor, where each sample was repeated for 3 times, the fermentation broth was replaced with blank culture medium as negative control, placing at 25° C. in the dark, and counting the nematode mortality at interval of 10 minutes (see FIG. 2).

The nematodes were second-stage juvenile of Meloidogyne incognita (Meloidogyne incongnita, J2).

Corrected Mortality (%)=(Treatment Mortality−Control Mortality)/(1−Control Mortality)×100

The BPY fermentation supernatant of strain AMCC 101084 showed 100% lethality to nematodes after 1.5 hours.

The fermentation supernatant of contrast strain 1 Bacillus subtilis AMCC100011, deposited at Shandong Agricultural Microbiological Culture Collection Center (from which the skilled in the art can purchase), had no lethal effect on nematodes under the same culture and nematicidal conditions.

The fermentation supernatant of contrast strain 2 Bacillus amyloliquefaciens AMCC100041, deposited at Shandong Agricultural Microbiological Culture Collection Center (from which the skilled in the art can purchase), showed 55% lethality to nematodes after 12 hours under the same culture and nematicidal conditions.

The fermentation supernatant of contrast strain 3 Bacillus licheniformis AMCC100161, deposited at Shandong Agricultural Microbiological Culture Collection Center (from which the skilled in the art can purchase), showed 43% lethality to nematodes after 12 hours under the same culture and nematicidal conditions.

The fermentation supernatant of contrast strain 4 Bacillus altitudinis AMCC101354, deposited at Shandong Agricultural Microbiological Culture Collection Center (from which the skilled in the art can purchase), showed 29% lethality to nematodes after 12 hours under the same culture and nematicidal conditions.

### Embodiment 3

The analysis of the nematicidal active substances of strain AMCC 101084 comprised the following steps.
(1) AMCC 101084 was inoculated into a BPY solid medium plate for activation culture at 37° C. for 12 hours to prepare an activated strain.
(2) The activated strain prepared in step (1) was inoculated into a BPY liquid medium for seed culture at 37° C. and 200 rpm for 12 hours to prepare a seed solution.
(3) The seed solution prepared in step (2) was inoculated into a BPY liquid medium with an inoculation amount of 7% by volume for liquid culture at 37° C. and 200 rpm for 12 hours to prepare a primary fermentation broth.
(4) The primary fermentation broth prepared in step (3) was inoculated into a BPY fermentation medium with an inoculation amount of 7% by volume for fermentation culture at 37° C. and 200 rpm for 48 hours to prepare a fermentation broth.
(5) The fermentation broth prepared in step (4) was placed in a high-speed refrigerated centrifuge at 12000 rpm, centrifuging at 4° C. for 10 minutes, sucking the supernatant, and performing SPME-GC-MS analysis under the following analysis conditions:

Aging of extraction head: under a temperature condition of 250° C., a flow rate of carrier gas was 3 mL/min, and the aging time was 30 minutes.

25 mL sample was added into a 50 mL head space bottle, and immediately sealed using a bottle cap with silicone rubber spacer, and placed in a water bath at 95° C. to process 30 minutes. The aged 50/30 μm DVB/CAR/PDMS solid phase micro-extraction head was inserted into the head space bottle without the end contacting with the sample, and kept at 95° C. for 30 minutes. The solid phase micro-extraction head with completed enrichment was taken out, and quickly inserted into an injection port of gas chromatograph, and desorbed at 250° C. for 6 minutes to complete injection.

Chromatographic conditions: Rtx-5MS column (60 m×0.32 mm×0.25 μm) was used for chromatographic separation. 99.999% helium was used as the carrier gas at a flow rate of 3 mL/min. The injection port, with a splitless injection mode, was 230° C. in temperature.

Mass spectral conditions: an ion source temperature was 220° C., ionization energy of EI ionization was 70 eV, an interface temperature was 250° C., an ion fragment scanning range was 35 to 500 m/z, and solvent delay time was 3 minutes.

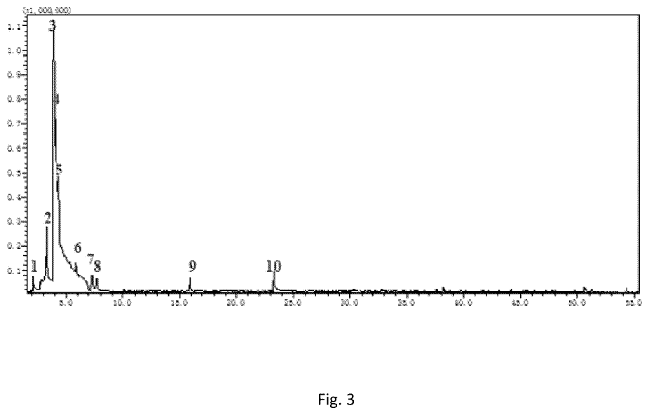

Heating procedure: the initial temperature was 50° C., then increased by 2° C./min to 120° C. after keeping 2 minutes, and then increased by 5° C./min to 200° C. to keep 1 minute. The volatile components were identified after comparison in NIST08, NIST08s and FFNSC1.3 databases with results shown in Table 2. FIG. 3 is total ion current of volatile substances, the ordinate being the peak value, and the abscissa being the time of peak emergence.

TABLE 2
Volatile components produced by AMCC 101084

| | Retention | Similarity | Peak |
|---|---|---|---|
| Compounds | Time (min) | (%) | number |
| 2,3-Butanedione | 2.736 | 96 | 1 |
| Acetic acid | 3.291 | 98 | 2 |
| Acetoin | 3.837 | 98 | 3 |
| 3-Methyl-1-butanol | 4.235 | 81 | 4 |
| 2-Isopropoxy ethylamine | 5.535 | 81 | 5 |
| 2,3-Butanediol | 5.829 | 96 | 6 |
| 3-Methyl-butanoic acid | 7.308 | 96 | 7 |
| 2-Methyl-butanoic acid | 7.701 | 96 | 8 |
| 2-Ethyl hexanol | 15.876 | 96 | 9 |
| n-Octanoic acid | 23.273 | 97 | 10 |

### Embodiment 4

Compound of Nematicidal Active Substances of AMCC 101084 and Pot Experiment for Disease Resistance

A standard of 10 volatile compounds detected in Embodiment 3 (purchased from Shanghai Aladdin Biochemical Technology Co. Ltd.) was compounded at a certain concentration as follows: mass percentages of 2,3-butanedione, acetic acid, acetoin, 3-methyl-1-butanol, 2-isopropoxy ethylamine, 2,3-butanediol, 3-methyl-butanoic acid, 2-methyl-butanoic acid, 2-ethyl hexanol and n-octanoic acid after compounding were, respectively, 0.3%, 0.2%, 0.7%, 0.3%, 0.8%, 0.4%, 0.2%, 0.2%, 0.5% and 0.05%, and the balance was water.

The pot experiment was carried out in the greenhouse of Shandong Agricultural University. The soil was collected from the experimental station of Shandong Agricultural University with a population density 15 nematodes per gram. The nematodes were identified as Meloidogyne incognita by molecular biology. A total of 4 treatments were set up: (1) CK, 80 mL sterile water+800 g nematode soil; (2) T1, 80 mL diluent of avermectin emulsifiable concentrate+800 g nematode soil, the diluent being a 2000 times diluent of 5% avermectin emulsifiable concentrate; (3) T2, 80 mL diluent of compound solution of nematicidal active substances+800 g nematode soil, the diluent being a 1000 times diluent of compound solution of the nematicidal active substances; (4) T3, 80 mL diluent of supernatant of fermentation broth II (prepared in Embodiment 2)+800 g nematode soil, the diluent being a 1000 times diluent of the supernatant of fermentation broth II. Tomatoes were transplanted after 3 days of treatment, with 6 replicates per treatment. According to the conventional management, the whole plant of tomatoes was sampled after 45 days of transplanting, and the number of root-knots and oocysts and population density of nematodes were counted, with a specific method referring to Affokpon et al. (Biocontrol potential of native Trichoderma isolates against root-knot nematodes in West African vegetable production systems, soil Biology and Biochemistry, 2011). The results were shown in Table 3.

TABLE 3
Pot experiment

| | | | Population |
|---|---|---|---|
| | Number of | | density of |
| | root-knots | Number of | nematodes |
| | (piece) | oocysts (piece) | (bars/g soil) |
| CK | 96.67 ± 4.63A | 65.33 ± 7.79A | 35.69 ± 3.23A |
| T1 | 7.67 ± 1.45C | 1.00 ± 0.58B | 2.90 ± 0.35C |
| T2 | 33.00 ± 3.61B | 10.00 ± 1.15B | 9.55 ± 0.54B |
| T3 | 35.05 ± 2.78B | 8.07 ± 0.21B | 9.77 ± 0.71B |

The results of pot experiment showed that tomatoes were transplanted after 3 days of treatment of nematode-infected soil with compound solution of nematicidal active substances of AMCC 101084. After 45 days of planting, the number of root-knots, population density of nematodes and oocysts decreased by 65.86%, 73.24% and 84.69%, respectively. Compared with the blank control, the number of root-knots, population density of nematodes and oocysts, treated by supernatant of fermentation broth II, decreased by 63.74%, 72.63% and 87.65%, respectively.

### Embodiment 5

Field Experiment of AMCC 101084

The experiment was carried out in the field of Changyi City, Weifang City, Shandong Province. The root-knot nematode disease was serious in ginger continuous cropping for many years.

There were 3 treatments in the experiment. (1) Microbial inoculum treatment: the concentration of fermentation broth I (prepared in Embodiment 2) was (0.2 to 1.0)×10⁹ CFU/mL, and the amount of mu was 20 L; after soil preparation and ridging, the microbial inoculum was diluted by 1000 times and uniformly sprayed into a ditch for planting ginger after 3 days of treatment. (2) Chemical pesticide treatment as positive control: thiazophosphine granules with 10 kg per mu were evenly sprinkled in the ditch. (3) Blank control (CK): no treatment was done. Each treatment was repeated 3 times, each treatment area being 30 m². Ramatsitsi and Dube (Post-infectional resistance in traditional leafy vegetable infected with root-knot nematodes. South African Journal of Botany, 131, 169-173) was used as reference for root staining method, root-knot number statistical method, and female number statistical method.

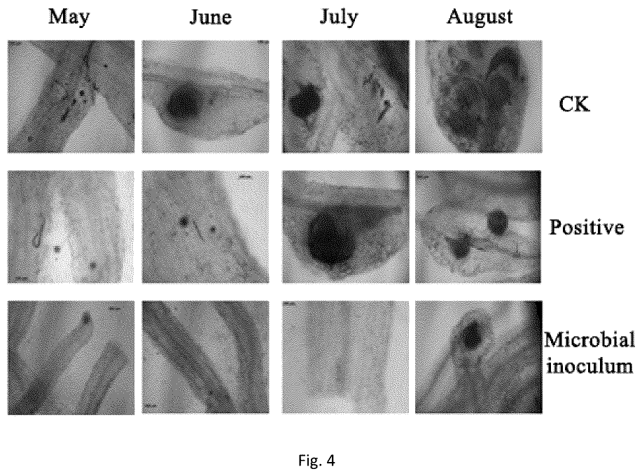

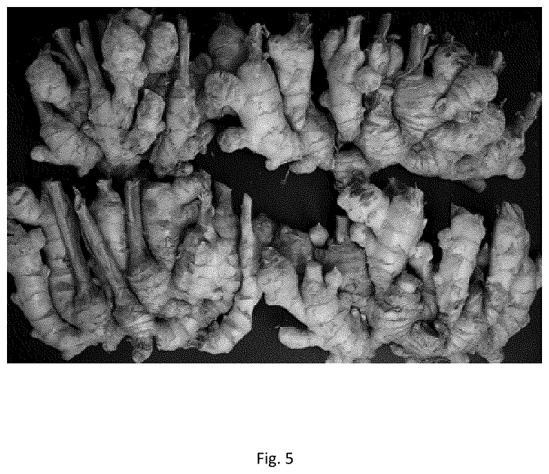

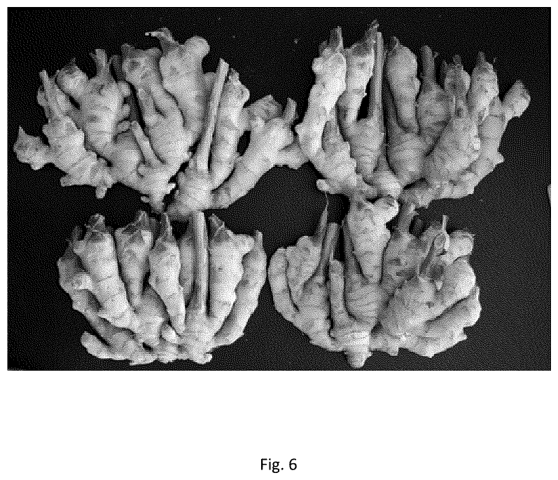

The results showed (see Table 4, FIG. 4) that compared with CK, the number of second-stage juvenile decreased by 93.68% in May, the number of female larvae decreased by 82.90% in August, the total number of root-knots (single root-knots+beaded root-knots) decreased by 65.71% in the period of root-knot nematode disease attack. The blank control group had a larger bark cracking area of ginger in harvest period (see FIG. 5), seriously affecting the product quality of ginger. The bark cracking area of ginger to which a microbial inoculum was applied significantly reduced (see FIG. 6), effectively improving the product quality of ginger. The microbial inoculum of the present invention has a significant control effect on root-knot nematodes.

It can be seen from FIG. 4 that the microbial inoculum of AMCC 101084 has a significant inhibitory effect on root-knot nematodes of ginger, which can effectively inhibit nematode infection and development and delay the development time of female nematode.

TABLE 4
Field experiment

| | Infection | Female | Single | Beaded | Disease |
|---|---|---|---|---|---|
| | J2s | number | root-knots | root-knots | index |
| CK | 31.67 ± 2.72A | 58.50 ± 4.51A | 56.33 ± 4.94A | 24.83 ± 3.17A | 0.70 |
| Positive | 4.83 ± 0.54B | 16.83 ± 2.06B | 32.50 ± 2.90B | 10.83 ± 1.70B | 0.23 |
| Microbial | 2.00 ± 0.37B | 10.00 ± 0.97B | 20.50 ± 1.67C | 7.33 ± 0.76B | 0.30 |
| inoculum | | | | | |

Note:
“J2s” are second-stage juvenile of root-knot nematodes.

In summary, fermentation supernatant of AMCC 101084 involved in the present invention shows 100% lethality to second-stage juvenile of Meloidogyne incognita in a nematicidal experiment, and the action time is short, with only 1.5 hours required to achieve the 100% lethality. After 3 days of treatment of nematode-infected soil with AMCC 101084 liquid microbial inoculum of the present invention, ginger is planted. In the later stage, the microbial inoculum is no longer applied, the concentration of the microbial inoculum is (0.2 to 1.0)×10⁹ CFU/mL, and the amount of mu is 20 L. Compared with the blank control, the bark cracking area of ginger to which the microbial inoculum is applied is significantly reduced, where the root-knot nematode disease of ginger is commonly known as ginger bark cracking disease, effectively improving the product quality of ginger. The control effect of the microbial inoculum of the present invention on root-knot nematodes is significant, and it is beneficial to popularization and application.

According to the types of active substances in the fermentation broth of AMCC 101084 of the present invention, the tomatoes are transplanted after 3 days of treatment of nematode-infected soil with the compound active substance compound solution. After 45 days of tomato planting, the number of root-knots decreases by 65.86% and population density of nematodes decreases by 73.24%.

What is claimed is:

1. Bacillus altitudinis AMCC 101084, deposited at China General Microbiological Culture Collection Center (CGMCC, No. 3, Yard 1, Beichen West Road, Chaoyang District, Beijing, China) on Aug. 16, 2018 with deposit number CGMCC No. 16308.

2. A culture method I of Bacillus altitudinis AMCC 101084 according to claim 1, comprising the following steps:
(1) activating and culturing Bacillus altitudinis AMCC 101084 on a solid medium plate to prepare an activated strain;
(2) inoculating the activated strain prepared in step (1) into a seed liquid medium for cultivation to prepare a seed solution;
(3) inoculating the seed solution prepared in step (2) into a fermentation liquid medium for cultivation to prepare a primary fermentation broth; and
(4) inoculating the primary fermentation broth prepared in step (3) into a fermentation liquid medium for cultivation to prepare a fermentation broth.

3. The culture method I according to claim 2, wherein in the step (1), the solid medium is a Luria-Bertani (LB) solid medium under an activation condition at 37° C. for 24 hours.

4. The culture method I according to claim 2, wherein in the step (2), the seed liquid medium is an LB liquid medium under a culture condition of 37° C. and 180 rpm for 18 hours.

5. The culture method I according to claim 2, wherein in the step (3), an inoculation amount is 5 to 10% by volume under a culture condition of 37° C. and 180 rpm for 10 hours.

6. The culture method I according to claim 2, wherein in the step (4), an inoculation amount is 5 to 10% by volume under a culture condition of 37° C. and 180 rpm for 24 hours.

7. The culture method I according to claim 2, wherein in the step (3) or (4), according to mass fraction, components of the fermentation liquid medium are as follows: 1.5% corn flour, 2.0% soybean meal, 2.1% bran, 0.03% KH₂PO₄, 0.01% K₂HPO₄, 0.01% CaCl₂), balance water, and 10 pH 7.0 to 7.2.

8. The culture method I according to claim 2, wherein in the step (4), a cell concentration of the prepared fermentation broth is (0.2 to 1.0)×10⁹ CFU/mL.

9. A culture method II of Bacillus altitudinis AMCC 101084 according to claim 1, comprising the following steps:
a. activating and culturing Bacillus altitudinis AMCC 101084 on an LB solid medium plate to prepare an activated strain;
b. inoculating the activated strain prepared in step a into a Beef Prptone Yeast (BPY) liquid medium for cultivation to prepare a seed solution; and
c. inoculating the seed solution prepared in step b into a BPY liquid medium for cultivation to prepare a fermentation broth.

10. The culture method II according to claim 9, wherein in the step a, an activation condition is at 37° C. for 12 hours.

11. The culture method II according to claim 9, wherein in the step b, a culture condition is at 37° C. and 200 rpm for 12 hours.

12. The culture method II according to claim 9, wherein in the step c, an inoculation amount is 1% and a culture condition is at 37° C. and 200 rpm for 48 hours.

13. The culture method II according to claim 9, wherein in the step b or c, components per liter of the BPY liquid medium are as follows:
ten grams of peptone, 5.0 g yeast extract, 5.0 g beef extract, 10.0 g glucose, 5.0 g sodium chloride, balance water, and pH 7.0.

14. The culture method II according to claim 9, wherein in the step c, a cell concentration of the prepared fermentation broth is (0.3 to 1.1)×108 CFU/mL.

15. Application of Bacillus altitudinis AMCC 101084 according to claim 1 in controlling root-knot nematodes.

16. The application according to claim 15, wherein Bacillus altitudinis AMCC 101084 is used for controlling root-knot nematodes of tomatoes and/or ginger.

17. Application of the fermentation broth prepared by the culture method of Bacillus altitudinis AMCC 101084 according to claim 2 in controlling root-knot nematodes.

18. The application according to claim 17, wherein the fermentation broth is used for controlling root-knot nematodes of tomatoes and/or ginger.

19. The application according to claim 17, wherein liquid of the fermentation broth without thallus is used for controlling root-knot nematodes.

20. The application according to claim 17, wherein the fermentation broth is applied at a dilution of 1000 times.

21. The application according to claim 17, wherein the fermentation broth is used for controlling root-knot nematodes of tomatoes and/or ginger in an amount of 20 L per mu.

22. Application of Bacillus altitudinis AMCC 101084 according to claim 1 in producing one or more of 2,3-butanedione, acetic acid, acetoin, 2-isopropoxy ethylamine, 2,3-butanediol, 3-methyl-butanoic acid, 2-methyl-butanoic acid, and n-octanoic acid.
